(19) United States
(12) Patent Application Publication
(10) Pub. No.: US 2016/0158381 A1
(43) Pub. Date: Jun. 9, 2016

(54) NOVEL NANOLIPOSOMES AND THEIR USE FOR THE TREATMENT OF AMYLOID PROTEIN DISEASES

(71) Applicants: DEPARTMENT OF VETERANS AFFAIRS, TECHNOLOGY TRANSFER PROGRAM, Washington, DC (US); MIDWESTERN UNIVERSITY, Downers Grove, IL (US)

(72) Inventors: Raymond Q. MIGRINO, WASHINGTON, DC (US); Volkmar WEISSIG, WASHINGTON, DC (US); Daniel A. FRANCO, Washington, DC (US); Seth TRURAN, Washington, DC (US)

(21) Appl. No.: 14/905,730

(22) PCT Filed: Jul. 17, 2014
(86) PCT No.: PCT/US2014/046988

§ 371 Date: Jan. 15, 2016

Related U.S. Application Data
(60) Provisional application No. 61/847,336, filed on Jul. 17, 2013.

(51) Int. Cl. A61K 47/48; C12N 15/113; A61K 9/127; A61K 38/17

(52) U.S. Cl. CPC A61K 47/48815; A61K 38/1761; A61K 38/1709; C12N 15/113; A61K 47/48246; A61K 38/1716; A61K 9/127; A61K 9/1271; A61K 9/1272; C12N 2310/14; C12N 2320/32

(57) ABSTRACT

Generally provided herein are methods, compounds, and compositions described useful for the treatment of light chain amyloidosis and other amyloid protein diseases.

### FIELD OF THE INVENTION

Generally provided herein are methods, compounds, and compositions described useful for the treatment of light chain amyloidosis and other amyloid protein diseases.

### BACKGROUND OF THE INVENTION

Immunoglobulin light chain amyloidosis (AL) and other amyloid protein misfolding diseases (such as Alzheimer's disease and Parkinson's disease) share common toxic pathophysiology in that the misfolded proteins (immunoglobulin light chain in AL or abeta protein in Alzheimer's disease) induce oxidative stress to vascular and non-vascular tissue leading to cell damage and organ dysfunction. There is currently no known direct treatment to protect cells against amyloid protein toxicity.

### SUMMARY OF THE INVENTION

Amyloid protein misfolding diseases are associated with vascular injury induced by amyloid proteins. Light chain amyloidosis (AL) is a protein-misfolding disease associated with high morbidity and mortality that involves plasma cell overproduction of amyloidogenic light chain proteins (LC) leading to multiorgan injury, particularly heart failure. Alzheimer's disease involves tissue injury from Aβ protein and is associated with vascular dysfunction.

This invention relates, in part, to the discovery that soluble/prefibrillar amyloid proteins such as LC or Aβ induce microvascular dysfunction in human arterioles. These findings are consistent with clinical observations of endothelial dysfunction in early and established disease. At the present time, chemotherapy±autologous stem cell transplantation to eradicate the plasma cells is the only treatment available to treat AL amyloidosis but it is associated with high treatment related mortality and cannot be given in many patients with advanced disease.

There is no current treatment for Alzheimer's disease and other amyloid protein misfolding disorders. Nanoliposomes (NL) are artificial phospholipid vesicles that can bind amyloid proteins such as light chains (in AL amyloidosis) or Aβ proteins (in Alzheimer's disease), pointing to their potential to modify injury by misfolded proteins. As described herein, it was discovered that nanoliposomes attenuate amyloid protein (LC and Aβ)-induced human arteriole endothelial dysfunction and protect against amyloid protein-induced human endothelial cell injury.

Other features and advantages of the present invention will become more readily apparent to those of ordinary skill in the art after reviewing the following detailed description and accompanying drawings.

### BRIEF DESCRIPTION OF THE DRAWINGS

The details of the present invention may be gleaned in part by study of the accompanying drawings, in which:

FIG. 1 provides illustrative embodiments in which nanoliposomes (NL) can protect cells against amyloid protein injury. The nanoliposomes used in FIGS. 1-6 (except FIG. 3B) are composed of phospholipids cholesterol, phosphatidylcholine and phosphatidic acid, but the composition can be varied to exploit electrochemical properties of lipid components. On the top panel, nanoliposomes are physically bound to amyloid proteins such as light chain proteins (LC), thereby reducing cell exposure to amyloid proteins. In middle panel, therapeutic agents such as clusterin (CLU) are non-covalently incorporated to nanoliposomes for cell, tissue and/or organelle targeted delivery of a therapeutic agent. In bottom panel, covalent binding or conjugation of one or more therapeutic agents to nanoliposomes to enhance cell, tissue and/or organelle delivery is done such as use of PEGylation utilizing PEG chains bearing functional groups at their distal end.

FIG. 2A is a western blot indicating reduction of soluble amyloid proteins (LC) when co-treated with nanoliposomes according to one embodiment of the invention. In this exemplary embodiment, soluble LC amyloid proteins (20 mcg/mL) were mixed at 1:1, 1:5 and 1:10 mass ratio to nanoliposomes and underwent ultracentrifugation. The supernatant soluble amyloid protein content was determined by anti-lambda Western blot. As illustrated in this embodiment, there is a significant reduction in free soluble amyloid proteins when mixed with nanoliposomes.

FIG. 2B illustrates an embodiment of the invention where nanoliposomes preserved endothelial function of human ex-vivo adipose arterioles exposed to soluble amyloid proteins.

FIG. 2C illustrates an embodiment of the invention where nanoliposomes protected endothelial cells against cell death (DNA fragmentation) when exposed to soluble amyloid proteins.

FIG. 3A exemplifies an embodiment of the invention where noncovalent mixing of nanoliposomes with human recombinant clusterin (300 ng/mL) resulted in reduced clusterin ELISA signal suggesting reduced clusterin epitope exposure to antibodies (and therefore clusterin incorporation). Co-treatment with triton (a detergent that destroys nanoliposomes) restores clusterin signal, suggesting that 54% of clusterin epitopes are incorporated into nanoliposomes.

FIG. 3B illustrates an embodiment of the invention where the incorporation of clusterin into nanoliposomes restored endothelial function of human ex-vivo adipose arterioles exposed to soluble amyloid proteins. Nanoliposomes were composed of cholesterol, phosphatidylcholine and stearyl triphenylphosphonium (STTP) and clusterin was incorporated by noncovalent means. In separate experiments we were successful in incorporating clusterin into nanoliposomes composed of cholesterol and phosphatidylcholine and co-treatment with light chain preserved human arteriole endothelial function (dilator response to acetylchonine: LC −37.8% versus baseline, LC+clusterin-nanoliposome +3.7% versus baseline, free clusterin without nanoliposome +3.1% versus baseline. We have also successfully PEGylated nanoliposomes and incorporated clusterin covalently through PEG arms demonstrating feasibility of covalent conjugation of compounds to nanoliposomes.

FIG. 4A illustrates and embodiment of the invention where one hour of exposure to Aβ42 caused endothelial dysfunction in abdominal adipose arterioles (to a similar degree as cadaver leptomeningeal arterioles). Co-treatment with nanoliposomes restored adipose arteriole endothelial function.

FIG. 4B illustrates that Aβ42 reduced endothelial cell NO gas production, an effect not seen with scrambled Aβ42, signifying vascular dysfunction may be related to reduced NO bioavailability.

FIG. 5A & FIG. 5B illustrate arteriole vasoreactivity and CD spectroscopy according to one embodiment of the invention. Dilator response to acetylcholine and papaverine was reduced with LC and restored with by NL. L-NAME abolished NL protective effect suggesting that one of the protective effects of NL is through enhancing nitric oxide bioavailability.

FIG. 5C illustrates that in one embodiment of the invention Far-UV-CD spectroscopy shows increased ellipticity (more negative value) at 211-220 nm wavelengths when AL-09 LC is mixed with NL signifying modification of protein populations with increased populations with enhanced beta-structure.

FIG. 5D illustrates that in one embodiment of the invention the thermal denaturation profile of ZL-09 was not changed by NL.

FIGS. 6A-D illustrate endothelial cell LC internalization and apoptotic injury in one embodiment of the invention. FIG. 6A and FIG. 6C display OG-stained LC was significantly reduced by co-treatment with NL. FIG. 6B and FIG. 6D display Hoecsht staining demonstrating reduced apoptotic injury with NL co-treatment.

FIGS. 7A-C illustrate a reduced dilator response to acetylcholine following LC treatment that was reversed by NLGM1 according to one embodiment of the invention. NLGM1 is a nanoliposome composed of cholesterol, phosphatidylcholine and GM1-ganglioside, illustrating the versatility of varying nanoliposome lipid/phospholipid composition to achieve protective effects against amyloid proteins. FIG. 7A: dilator response to acetylcholine 10⁻⁴M: Control-88.1±3.4%, LC+NLGM1-87.6±5.4%, LC-53%±3.8% m p≦0.001 LC vs. Control or LC+NLGM1. FIG. 7B: NLGM1 reduced endothelial cell LC internalization. FIG. 7C: AFM confirmed physiochemical interaction between LC and NLGM1: height NLGM1 1.85±0.14, LC 1.01±0.03, LC+NLGM1 2.76±0.2 nM, p<0.05 ANOVA and each 2 way comparison.

FIGS. 8A & 8B illustrate that Nanoliposomes attenuate β-amyloid induced human microvascular endothelial dysfunction according to one embodiment of the invention. FIG. 8A illustrates that dilator responses to acetylcholine and papaverine were reduced by Aβ and partially restored by NLPA (nanoliposomes composed of cholesterol, phosphatidylcholine and phosphatidic acid) according to one embodiment of the invention (dilation to acetylcholine 10⁻⁴M: Control-92.9±1.6%, Aβ+NLPA-83.2±5.6%, Aβ-61.5±5.7%, p<0.05 Aβ vs Control or Aβ+NLPA). FIG. 8B illustrates that NO production in HUVECs was reduced by Aβ and partially restored by NLPA according to one embodiment of the invention.

FIGS. 9A-C illustrate that phosphatidic acid containing nanoliposomes reduce AL amyloidosis light chain protein internalization, cell membrane permeability and toxicity in endothelial cells according to various embodiments of the invention. FIGS. 9A and 9B illustrate that LC caused cell membrane disruption manifested as increased intracellular calcein (the calcein used being cell membrane impermeant) and increased cell death (manifested through propidium iodide fluorescence) according to these embodiments of the invention. FIGS. 9A and 9B illustrate that NLPA reduced these effects and decreased LC internalization (using fluorophore tagged LC with fluorescence relative to control Control: 1±0, LC:3.64±0.8; LC+NLPA:1.95±0.6, p<0.05 LC vs. C, LC vs. LC+NLPA). FIG. 9C illustrates the AFM confirmed biochemical interaction between NLPA and LC according to one embodiment of the invention.

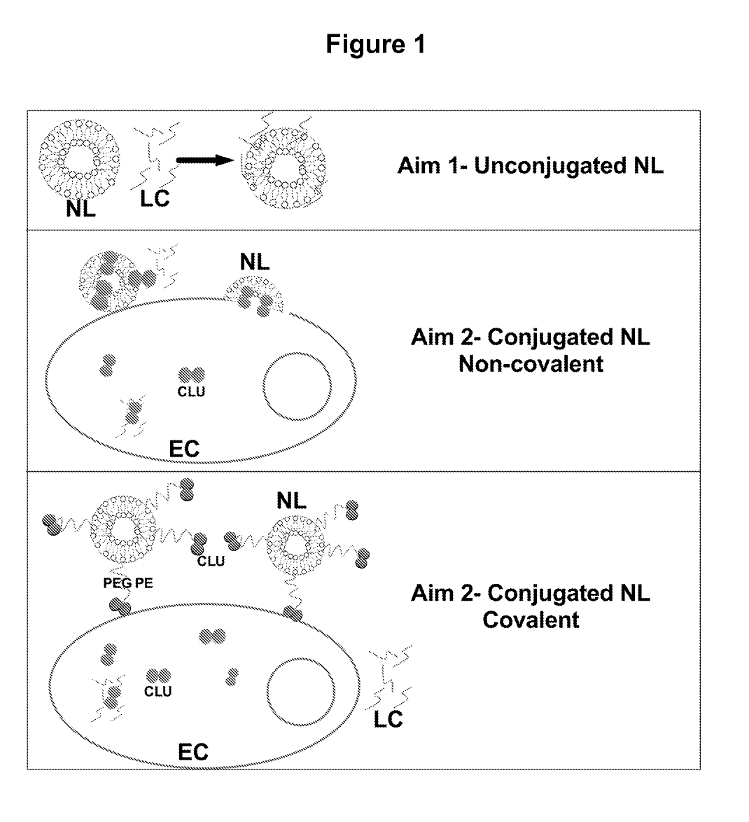

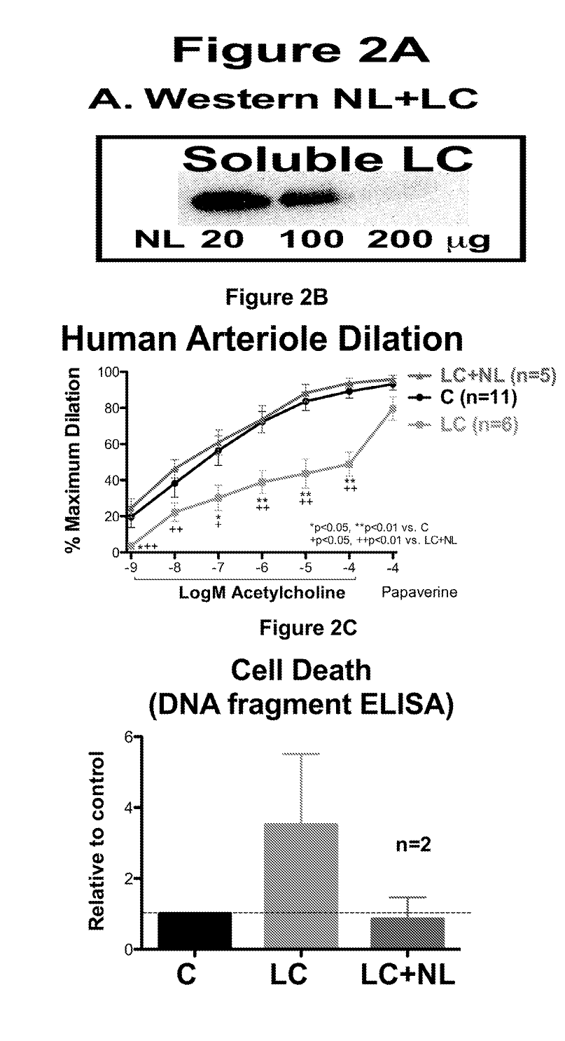

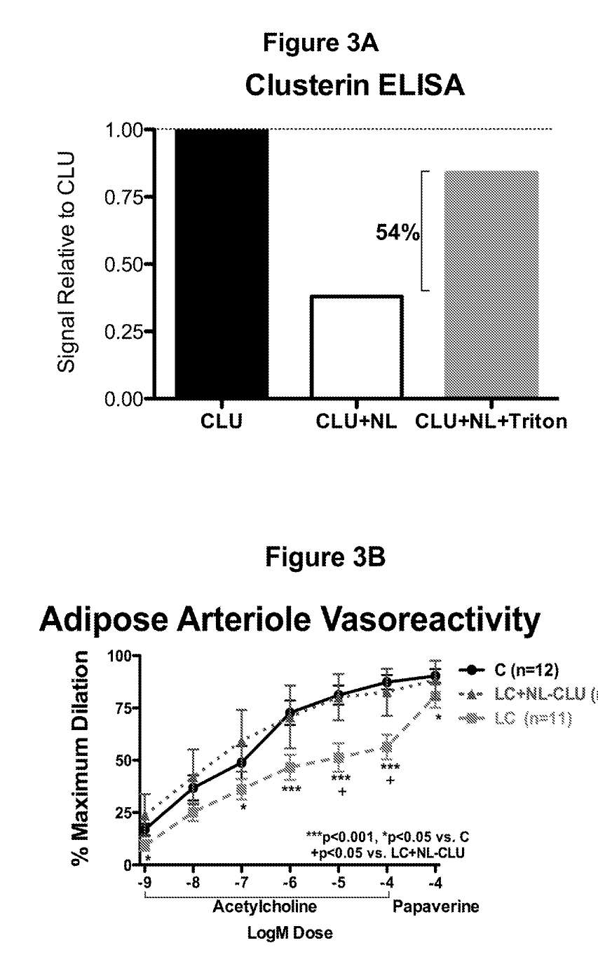

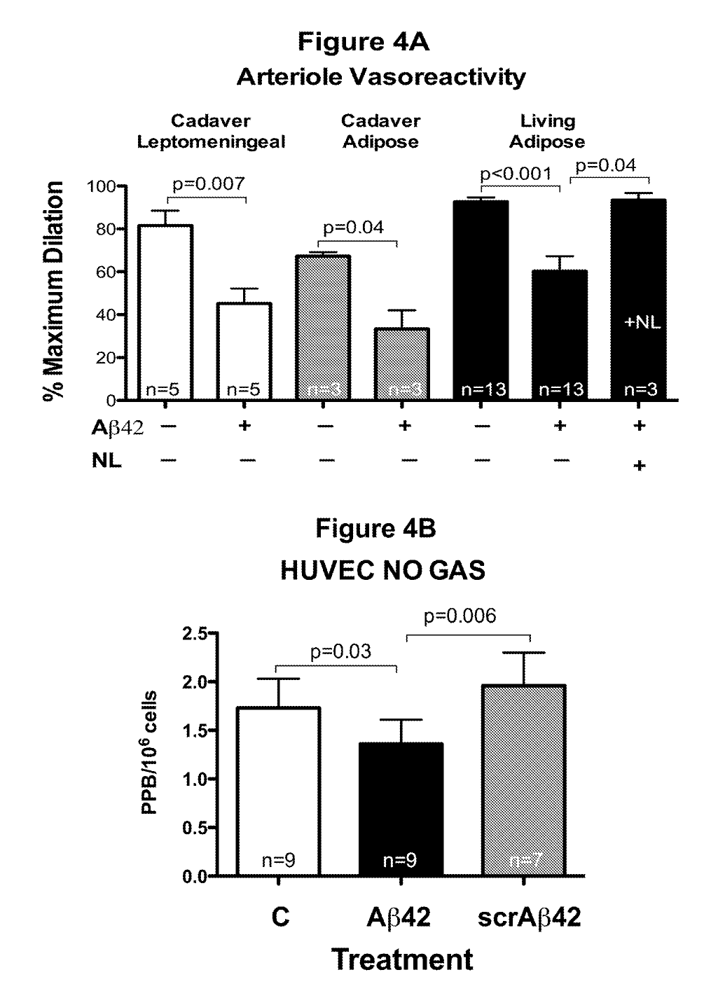

### DETAILED DESCRIPTION OF ILLUSTRATIVE EMBODIMENTS

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### Exemplary Terms

As used herein, the terms “comprising,” “including,” and “such as” are used in their open, non-limiting sense.

The term “about” is used synonymously with the term “approximately.” Illustratively, the use of the term “about” indicates that values slightly outside the cited values, i.e., plus or minus 0.1% to 10%, which are also effective and safe. Such dosages are thus encompassed by the scope of the description and claims reciting the terms “about” and “approximately.”

### Exemplary Compositions

Generally speaking, one will desire a composition that provides the therapeutic effect desired when administered to a subject. Determination of these parameters is well within the skill of the art. These considerations are well known in the art and are described in standard textbooks.

Provided herein are novel nanoliposomes. In specific embodiments, the novel nanoliposomes attach to amyloid proteins. In some embodiments of the invention, the novel nanoliposomes reduce the amount of soluble amyloid proteins. In other embodiments, the novel nanoliposomes preserve endothelial function of human arterioles when co-treated with light chain amyloid proteins. In yet other embodiments, the novel nanoliposomes protect endothelial cells against cell damage when co-treated with amyloid proteins such as light chain proteins.

In some embodiments the nanoliposomes comprise a combination of lipids/phospholipids that attach to amyloid proteins. Non-limiting examples of lipids/phospholipids that attach to amyloid proteins include cholesterol, phosphatidylcholine, phosphatidic acid, sphingomyelin, GM1 ganglioside and STTP.

In various embodiments, the nanoliposomes comprise additional cargoes. Exemplary additional cargoes useful in the present invention include, but are not limited to, apolipoproteins, including but not limited to clusterin, apolipoprotein A1, and E; peptides; antibodies; nucleic acids such as siRNA; aptamers; and mitochondrially targeted antioxidants, including but not limited to physiological nonenzymatic antioxidants such as vitamin E, ascorbic acid, glutathione and NADPH, nonenzymatic naturally occurring antioxidants of herbal origin such as resveratrol, enzymatic antioxidants such as superoxide dismutases and catalase as well as synthetic antioxidants such as modified tocopherols, derivatives of stobadine and dihydropyridines and modified enzymatic antioxidants. Although specific cargoes are recited herein, they are to be understood to be but examples of therapeutic agents that can be incorporated into the nanoliposomes.

In some embodiments, the nanoliposomes are less than about 100 nm, or less than about 75 nm, or less than about 50 nm. In some embodiments, the nanoliposomes are between about 1 nm and 50 nm, or between about 1 nm and 75 nm or between about 1 nm and 100 nm.

### Exemplary Methods of Treatment

Generally provided herein are methods and compositions useful for the treatment of light chain amyloidosis and other amyloid protein diseases.

Non-limiting examples of amyloid protein diseases include light chain amyloidosis; Alzheimer's disease; diabetes mellitus; Parkinson's disease; amyotrophic lateral sclerosis; Huntington's disease; AA amyloidosis; ATTR amyloidosis; hemodialysis associated amyloidosis (beta 2 microglobulin); prion diseases including but not limited to Creutzfeld Jakob disease, bovine spongiform encephalopathy, and scrapie; finish type amyloidosis; cerebral amyloid angiopathy; prolactinoma; familial corneal amyloidosis; senile amyloid of atria; medullary carcinoma of thyroid; and some amyloid forms of atherosclerosis.

In various embodiments, tissue function, such as endothelial function of human arterioles, is preserved by co-treating with nanoliposomes. In other embodiments, human cells such as endothelial cells are protected against cell damage by co-treating with nanoliposomes.

In various embodiments, clusterin (also known as apolipoprotein J, a type of chaperone protein) therapeutic agents are incorporated into nanoliposomes resulting in reduced clusterin epitope exposure and achieve enhanced bioavailability and tissue targeting. In some embodiments, the conjugation of clusterin with nanoliposomes preserves endothelial function of human arterioles exposed to amyloid proteins (such as light chain).

### EXAMPLES

Nanoliposomes, both unconjugated and conjugated with chaperone protein (clusterin) cargo, are developed to reverse amyloid protein (including but not limited to Aβ42, Aβ40 and LC) induced endothelial dysfunction in human arterioles/blood vessels/tissues/cells. In addition, mechanisms by which nanoliposomes exert protective effects include effects on protein stabilization and physico-chemical sequestration versus direct cellular protective effects.

### Example 1

### Unconjugated Nanoliposomes that Bind to Aβ42 and Aβ40 Proteins

Nanoliposomes using various ratios and compositions such as various combinations of cholesterol, phosphatidylcholine, sphingomyelin, phosphatidic acid and cardiolipin are prepared to maximize binding affinity to amyloid proteins such as abeta.

Nanoliposomes phospholipid compositions (with varying electrostatic charges) are varied to produce nanoliposomes with the most efficient binding or protein stabilization of amyloid proteins such as Aβ42 and Aβ40. These nanoliposomes are then tested for efficacy in preventing amyloid protein toxicity in Example 2.

### Example 2

### Protective Effect of Unconjugated Nanoliposomes Against AR-Induced Human Arteriole Vascular Dysfunction

The nanoliposomes protection against amyloid protein induced human brain or peripheral vessel or microvascular dysfunction is quantified while testing key potential mechanisms of protection.

It is then determined whether nanoliposomes protect by physicochemical sequestration/protein stabilization or whether nanoliposomes have additional direct cellular protective effects (such as enhancing NO bioavailability, reducing oxidative stress and vascular inflammation/apoptosis among others).

### Example 3

### Functionalized Nanoliposomes by Conjugation with Clusterin

Nanoliposomes are functionalized by conjugation with clusterin (a chaperone protein critical in Alzheimer Disease with dual roles of transporting abnormal proteins and promoting cell survival) to promote intracellular clusterin delivery and to test NL-clusterin's protective effect against amyloid protein induced arteriole dysfunction while testing potential mechanisms of protection.

Clusterin incorporation into nanoliposomes are optimized using noncovalent methods and by covalent methods such as PEGylation and other covalent techniques and quantified by ELISA and gradient ultracentrifugation.

### Example 4

### Quantification of Functionalized Nanoliposomes

The clusterin-conjugated nanoliposomes protection against amyloid protein induced microvascular dysfunction is quantified while testing potential mechanisms of protection.

Arterioles exposed to amyloid proteins such as abeta are co-treated with NL-clusterin to assess endothelial function, oxidative stress and apoptosis. It is then determined whether or not clusterin has extra/intracellular chaperone properties that reduce tissue exposure to amyloid proteins. NL-clusterin effects on reducing cell death through bax stabilization or other mechanisms are examined.

### Example 5

### Quantification of Functionalized Nanoliposomes

Amyloidogenic light chain proteins were purified from urine collected from 2 male patients with cardiac involvement (58±15 years old, lambda type) by dialysis, size exclusion and Affigel blue filtration and lyophilization and verified using anti-human lambda ELISA and Western blot.

In addition to patient-derived amyloidogenic light chain proteins, human recombinant full length light chain protein (AL-09) was also used. AL-09 was derived from a κ1-LC from a cardiac AL patient (protein sequence in GenBank, accession AF490909).

Nanoliposomes:

Nanoliposomes (phosphatidylcholine/cholesterol/phosphatidic acid 70/25/5 molar ratio; 20 mg lipid/ml; Avanti Polar Lipids, Alabaster Ala.) were prepared. The lipid mixture was dissolved in chloroform then organic solvent was removed by rotary evaporator. After adding 5 mM HEPES (pH 7.4) to dry lipid film, the sample was probe sonicated (Sonic Dismembrator 100, Fischer Scientific, power output ˜10 Watts, 30 minutes). Nanoliposome size and zeta potential (Coulter N4 Submicron Particle Size Analyzer) were 29±6 nm and −11.1 mV, respectively.

Arteriole Vasoreactivity:

9 male subjects (64±3 years old) without AL/diabetes/vascular disease undergoing routine abdominal surgery donated subcutaneous adipose tissue obtained by surgeons. Arterioles (˜100-200 μM diameter) were isolated, cannulated and pressurized (60 mmHg). Baseline/control vasoreactivity was performed following preconstriction with endothelin-1 (60% baseline diameter) and successive administration of acetylcholine (10-9-10-4M) to measure endothelium-dependent dilation and papaverine (10-4M) to measure smooth-muscle dependent dilation (videomicrometer). After washing, vessels were exposed to LC (20 μg/mL, physiologic concentration of circulating LC in patients) with or without NL (1:10 LC:NL mass ratio) for 1 hour and a second vasoreactivity response performed. In additional arterioles, LC+NL were co-treated with L-NG-nitroarginine methyl ester (L-NAME, 5 mmol, Sigma-Aldrich).

Circular Dichroism (CD) Spectroscopy:

AL-09 secondary structure was characterized by Far-UV-CD spectrum (260-200 nm). AL-09 (10 μM) was mixed 1:1 with NL and incubated (30 min) before spectrum was obtained. Thermal denaturation curves of AL-09±NL were monitored at the maximum β-sheet signal (217 nm) and ellipticity was measured from 14-80° C. (n=3).

Oregon Green (OG) Labeling:

Labeling of LC was achieved using OG-488 protein labeling kit (Molecular Probes, Eugene Oreg.). OG labeling of LC was achieved using OG 488 protein labeling kit (Molecular Probes, Eugene Oreg.). 50 μL of 1M bicarbonate was added to LC (1 mg) and added to 1 vial of OG reactive dye, stirring the mixture for 1 hour at room temperature. Labeled protein was purified by passing the mixture through a column with purification resin while adding elution buffer until the labeled protein has been eluted.

Using handheld UV lamp, the first band representing labeled protein was collected while slower moving band consisting of unincorporated dye was discarded. The degree of OG protein labeling was determined by measuring the absorbance of the conjugate solution at 280 nm and 496 nm. The concentration of the protein in the sample was calculated as:

$$Protein Concentration ( M ) = \frac{[ {A}_{280} - ( {A}_{496} \times 0.120 ] \times dilution factor}{203 \text{,} 000}$$

Where 203,000 cm⁻¹M⁻¹ is the molar extinction coefficient of a typical IgG and 0.12 is the correction factor to account for absorption of the dye at 280 nm. The degree of labeling was calculated as:

$$Moles dye per mole protein = \frac{{A}_{496} \times dilution factor}{70 \text{,} 0000 \times protein concentration ( M )}$$

The degree of labeling was 3.6 M dye/M protein.

Endothelial Cell LC Entry and Death:

Human aortic endothelial cells (HAEC, Lonza, Portsmouth N.H.) were exposed to OG-labeled LC (20 μg/mL)±NL (LC:NL 1:10 mass ratio) for 24 hours. Cells were fixed (4% formaldehyde) and stained using 1 μM Hoechst-33258 (Sigma-Aldritch). Confocal microscopy was performed (Zeiss 710 laser confocal microscope) using 488 nm excitation/515-535 emission (OG) or 405 nm excitation/415-460 nm emission (Hoechst). OG signal was compared versus control (HAEC exposed to OG-labeled LC for 10 minutes, ImageJ National Institutes of Health, Bethesda Md.). Apoptosis was determined using Hoechst staining by measuring the percentage of cells with dense concentrated granular nuclear fluorescence; cells were considered viable if they had diffused nuclear fluorescence. Measurement was performed by reader blind to treatment allocation.

Data/Statistical Analyses:

Data is expressed as means±standard error of means. Baseline control and post-treatment dilator response were compared using paired Student's t-test. Overall acetylcholine dilator response was analyzed by deriving the log effective concentration 50% (log EC50) using nonlinear regression and variable slope and least squares fit. For group analyses, one-way or two way analysis of variance with Bonferroni post-test were utilized (GraphPad Prism 5.0, San Diego Calif.).

Results:

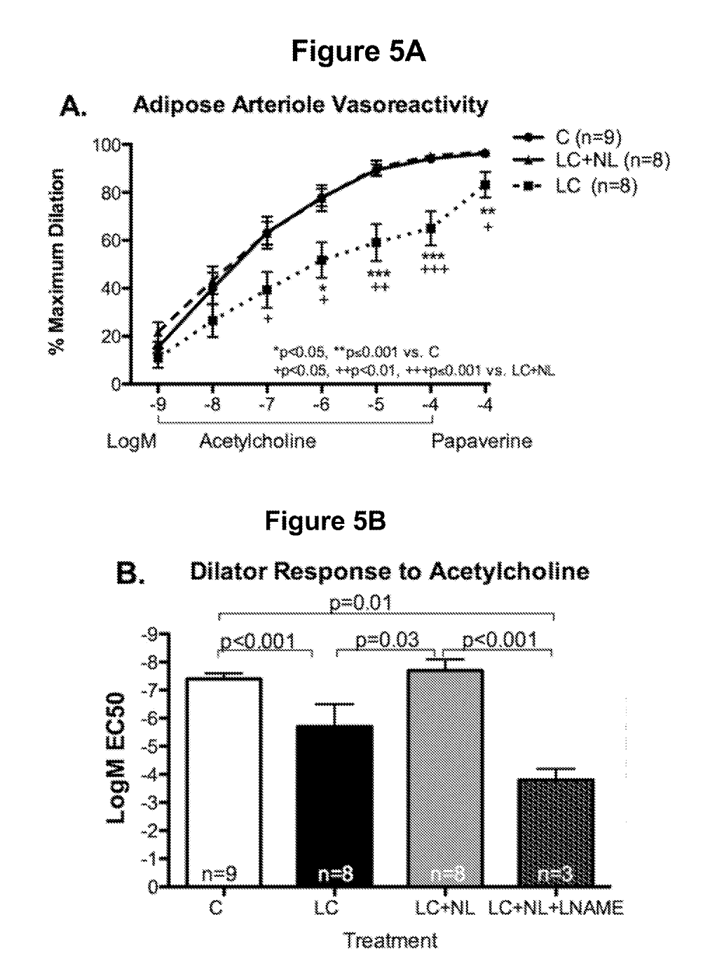

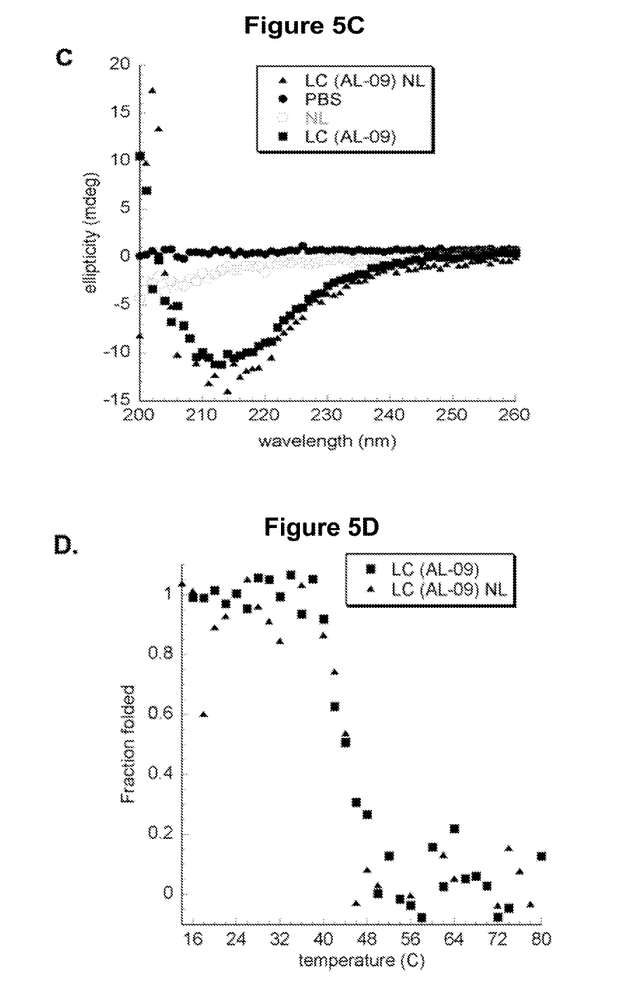

LC reduced dilation to acetylcholine and, to a lesser extent, papaverine in adipose arterioles (FIG. 5A and FIG. 5B). Nanoliposome co-treatment fully restored dilator responses. Nanoliposome protective effect was reversed by nitric oxide synthase inhibitor L-NAME.

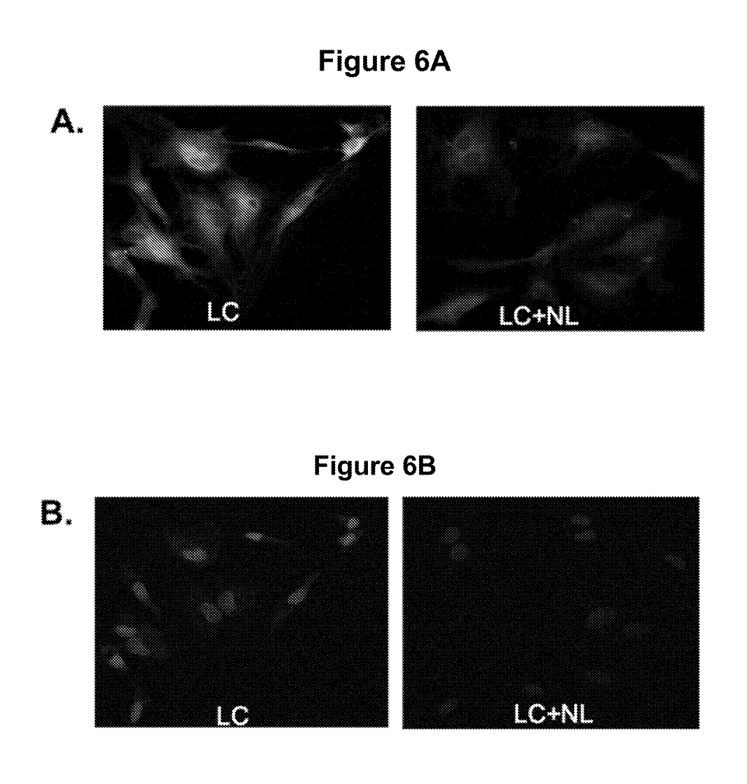

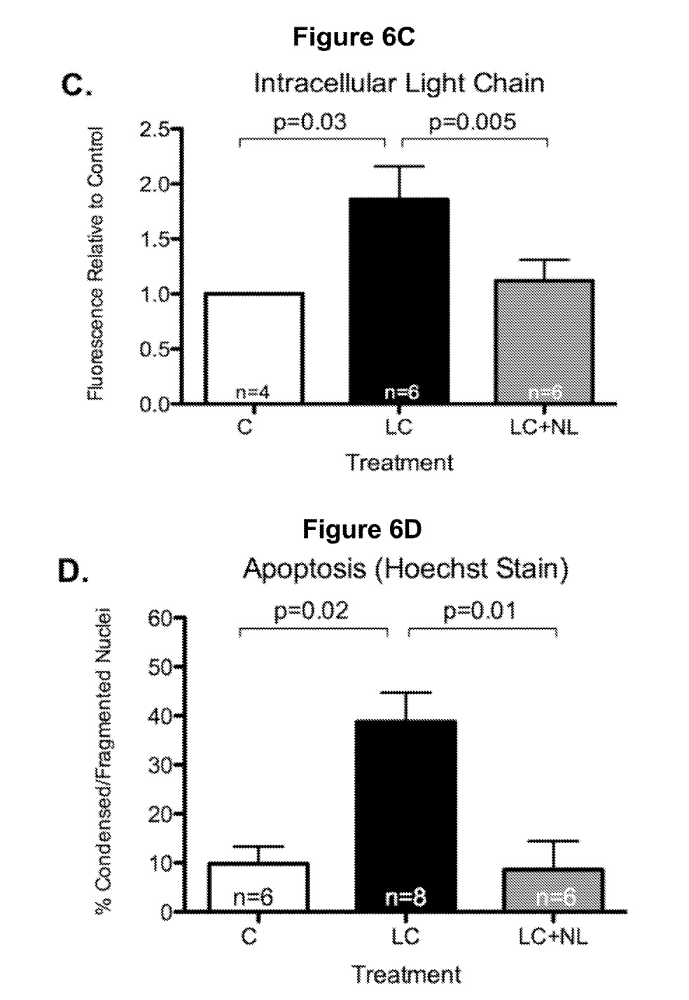

Nanoliposomes increased AL-09 LC ellipticity at 211-220 wavelength (n=3, p<0.001) suggesting increased AL-09 beta-sheet structure (see FIG. 5C). AL-09 thermal denaturation profile was not affected by NL (see FIG. 5D). Nanoliposomes decreased HAEC LC internalization (see FIG. 6A and FIG. 6C) and reduced apoptotic death (see FIG. 6B and FIG. 6D).

### Example 6

### GM1 Ganglioside-Containing Nanoliposomes Protect Against AL Amyloid Light Chain-Induced Human Microvascular Endothelial Dysfunction

AL amyloidosis involves multiorgan tissue damage from amyloid-forming light chain proteins (LC). As described herein, the potential of phosphatidic acid-containing nanoliposomes in mitigating LC-induced vascular dysfunction. In this example, GM1 ganglioside-containing liposomes were shown to have affinity to bind amyloid proteins rendering them useful in reducing LC injury.

GM1 ganglioside-containing nanoliposomes (NLGM1) will protect against LCinduced human microvascular endothelial dysfunction.

Ex-vivo subcutaneous adipose arterioles from subjects without known vascular disease/AL were cannulated and dilator response to acetylcholine and papaverine were measured at baseline (control) and following 1-hour exposure to LC (20 μg/mL, purified from urine of 2 AL patients)±NLGM1 (1:10 LC:NLGM1 ratio; 70% cholesterol, 25% phosphatidylcholine, 5% GM1 ganglioside).

Human umbilical vein endothelial cells (HUVECs) were exposed for 24 hours to LC incorporated with Oregon green fluorophore±NLGM1 and LC internalization was measured by confocal microscopy. Atomic force microscopy was used to determine interaction between LC (recombinant AL-09 derived from AL subject) and NLGM1.

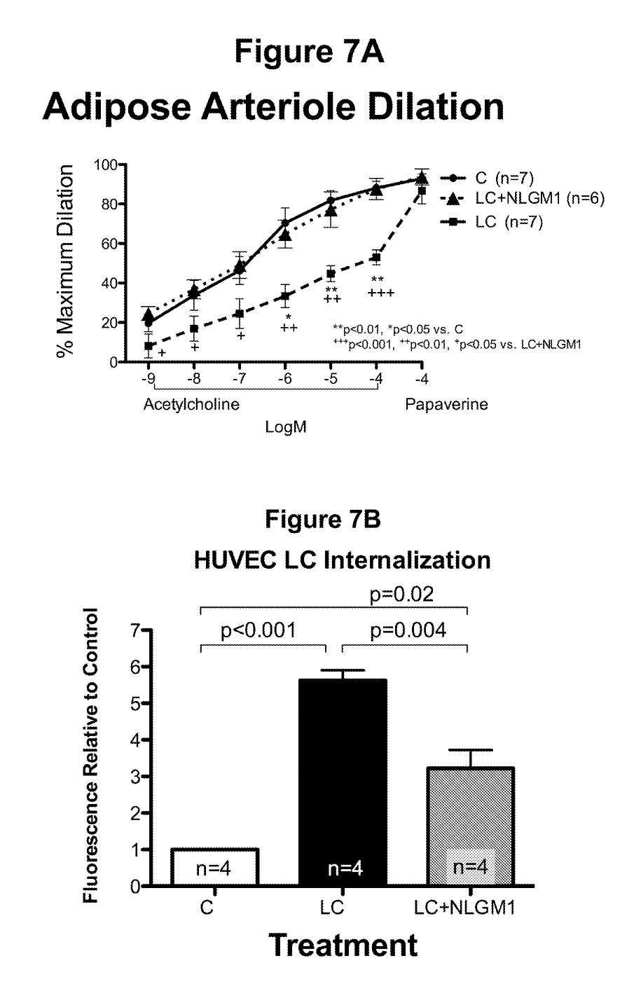

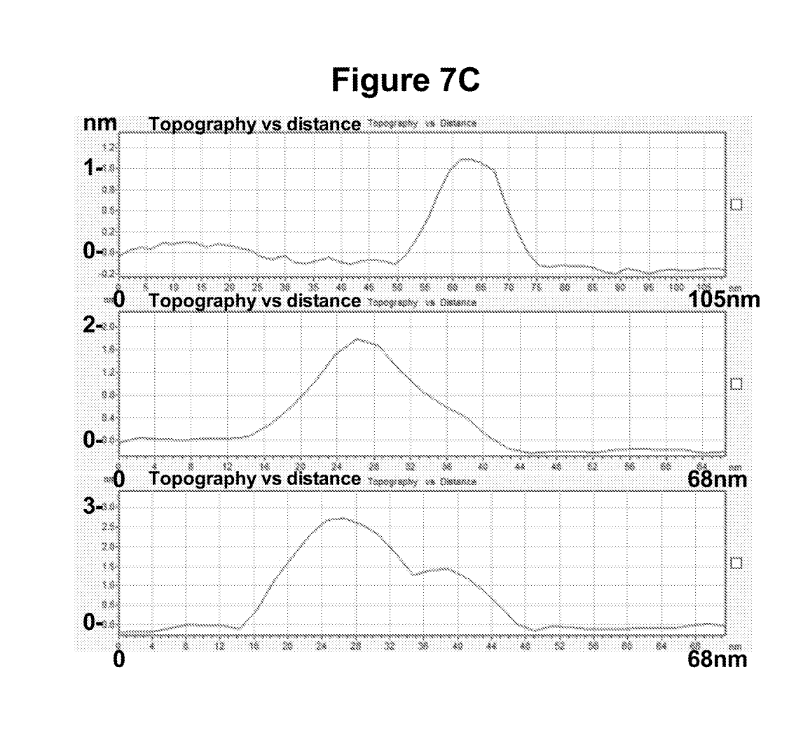

As illustrated in FIGS. 7A-C, there was reduced dilator response to acetylcholine following LC treatment that was reversed by NLGM1 (see FIG. 7A: acetylcholine 10-4M dilation: C-88.1±3.4, LC+NLGM1-87.6±5.4, LC-53.0±3.8%, p≦0.001 vs. C/LC+NLGM1).

NLGM1 reduced endothelial cell LC internalization (see FIG. 7B).

AFM confirmed physicochemical interaction between LC and NLGM1 (see FIG. 7C: height NLGM1 1.85±0.14, LC 1.01±0.03, LC+NLGM1 2.76±0.2 nM, p<0.05 ANOVA and each 2 way comparison).

Conclusion:

LC caused endothelial dysfunction in human arterioles that was reversed by co-treatment with NLGM1, potentially by interacting with LC and reducing endothelial cell LC internalization. NLGM1 has potential as a new therapeutic agent for AL.

### Example 7

### Nanoliposomes Attenuate β-Amyloid Induced Human Microvascular Endothelial Dysfunction

One of the poorly-understood but increasingly-recognized mechanisms of Alzheimer's disease (AD) is vascular dysfunction. To date there remains no effective treatment. We showed that nanoliposomes can prevent AL light chain-induced vascular dysfunction, a similar protein-misfolding disorder.

Without being bound by theory, the endothelial dysfunction induced by Aβ42 peptide, one of the amyloid proteins involved in AD, are attenuated by nanoliposomes.

Human abdominal subcutaneous arterioles were isolated from subjects without vascular disease, pressurized and constricted with endothelin-1. Baseline (control) dilation response was measured following acetylcholine (10_9-10-4M) and papaverine (10-4M) exposure; after washing, arterioles were exposed to Aβ42 (2 μM)±nanoliposomes (NLPA, 70% cholesterol, 25% phosphatidylcholine, 5% phosphatidic acid, 1:10 Aβ:NLPA mass ratio) and dilator response was measured.

Human umbilical vein endothelial cells (HUVECs) were exposed to Aβ42±NLPA or control for 1-hour, acetylcholine (10-4M) was administered at 45 minutes and nitric oxide (NO production) was measured using DAF-2 diacetate fluorescence (1 hour compared to baseline fluorescence).

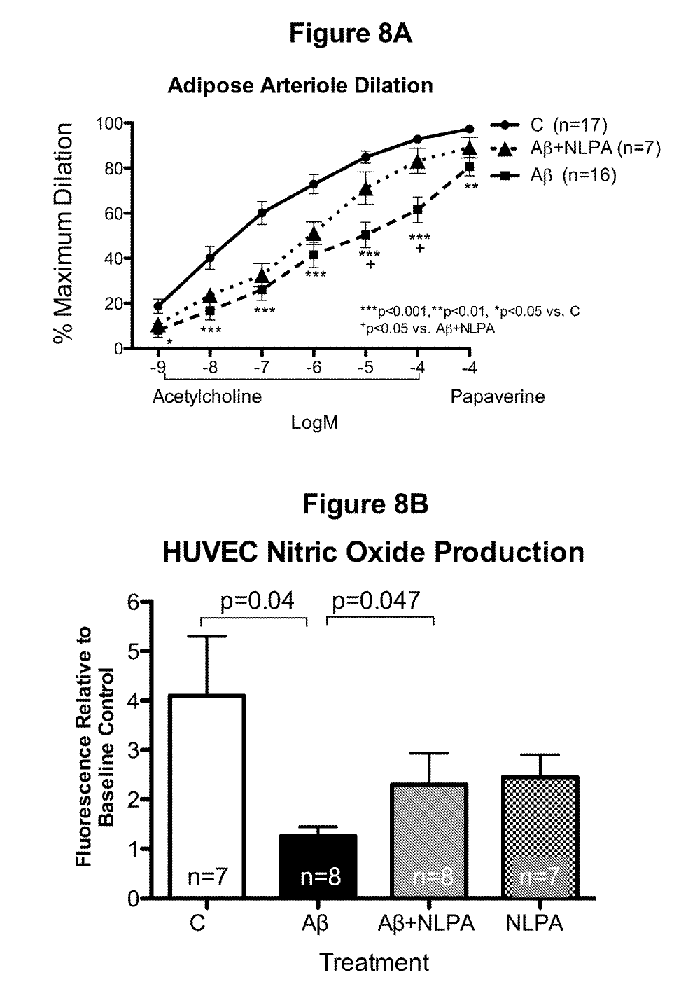

As illustrated in FIGS. 8A-B, Dilator responses to acetylcholine and papaverine were reduced by Aβ and partially restored by NLPA (see FIG. 8A: dilation to acetylcholine 10-4M: C-92.9±1.6, Aβ+NLPA-83.2±5.6, Aβ-61.5±5.7%, p<0.05 vs C and Aβ+NLPA). NO production in HUVECs was reduced by Aβ and partially restored by NLPA (see FIG. 8B).

Conclusion:

Acute Aβ42 exposure resulted in human arteriole endothelial dysfunction that was attenuated by co-treatment with nanoliposomes likely by enhancing nitric oxide bioavailability. Nanoliposomes represents a novel therapy for AD and use of easily accessible subcutaneous adipose tissue represents a practical human model to test novel therapies in AD.

### Example 8

### Phosphatidic Acid Containing Nanoliposomes Reduce AL Amyloidosis Light Chain Protein Internalization, Cell Membrane Permeability and Toxicity in Endothelial Cells

AL or light chain amyloidosis is associated with poor outcomes but the bases for tissue injury remain poorly understood. As described herein, phosphatidic acid-containing nanoliposomes (NLPA, 70% cholesterol, 25% phosphatidylcholine and 5% phosphatidic acid) restore endothelial function in human arterioles exposed to AL amyloidosis light chain proteins (LC).

Without being bound my theory, LC impair human umbilical vein endothelial cell (HUVECs) permeability and viability and NLPA mitigates these effects.

HUVECs were exposed to vehicle or LC (20 μg/mL, derived from urine LC of AL subject with cardiac amyloidosis)±NLPA (1:10 LC:NLPA mass ratio) for 1 hour followed by exposure to calcein (cell-membrane impermeant fluorophore), and for 24 hours followed by exposure to propidium iodide (cell death marker).

HUVECs were also exposed for 24 hours to vehicle, Oregon green labeled LC±NLPA and LC internalization was measured by confocal microscopy. Atomic force microscopy (AFM) was used to determine interaction between AL-09 LC (recombinant LC from cardiac amyloid subject) and NLPA.

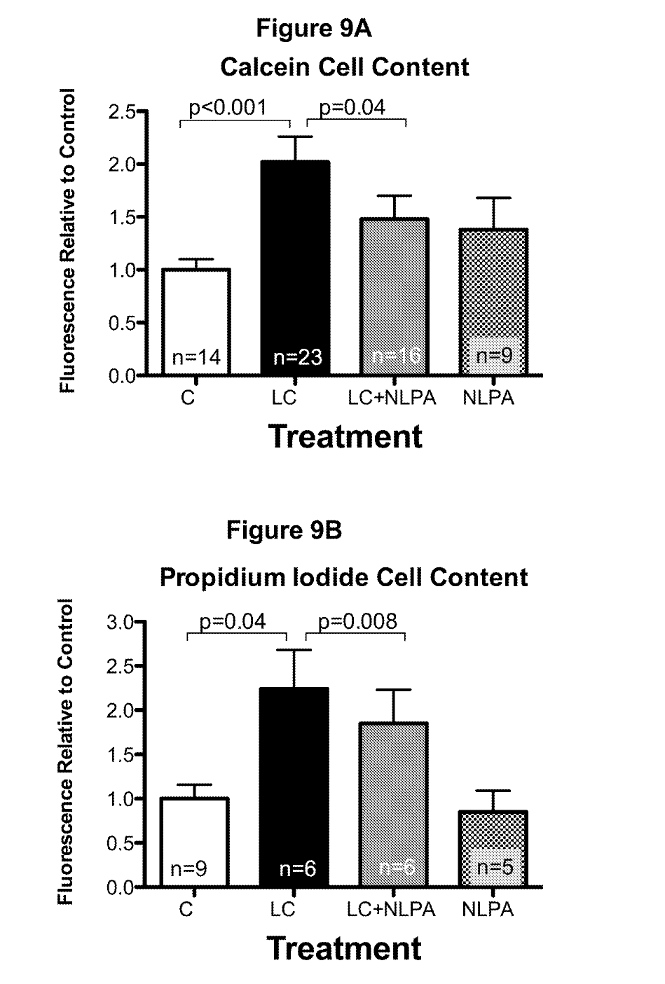

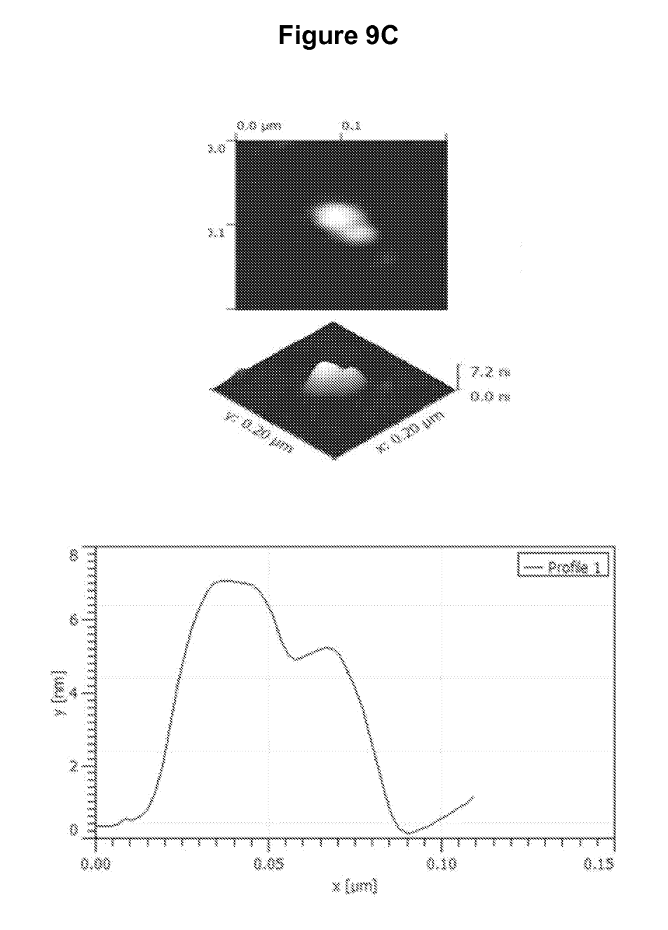

As illustrated in FIGS. 9A-C, LC caused cell membrane disruption. Specifically, increased calcein (see FIG. 9A) and increased cell death (see FIG. 9B). NLPA reduced these effects and decreased LC internalization fluorescence relative to control C:1±0, LC:3.64±0.8; LC+NLPA:1.95±0.6, p<0.05 LC vs. C, LC vs. LC+NLPA). AFM confirmed biochemical interaction between NLPA and LC (see FIG. 9C).

Conclusion:

NLPA interacted with LC leading to reduced LC cell internalization and partial protection against LC induced impaired cell permeability and cell death. Membrane disruption may be a novel mechanism of AL injury and potential pathway of nanoliposome protection.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A nanoliposome composition comprising lipids or phospholipids that attach to amyloid proteins.

2. The composition of claim 1, wherein the lipids or phospholipids that attach to amyloid proteins are composed of cholesterol, phosphatidylcholine, phosphatidic acid, sphingomyelin, GM1 ganglioside, STTP, other phospholipids or a mixture thereof.

3. The composition of claim 1, further comprising one or more cargoes.

4. The composition of claim 3, wherein the one or more cargoes comprise apolipoproteins, peptides, antibodies, nucleic acids, aptamers, mitochondrially targeted antioxidants or mixtures thereof.

5. The composition of claim 4, wherein the apolipoprotein is clusterin, apolipoprotein A1, or E.

6. The composition of claim 4, wherein the nucleic acid is siRNA.

7. The composition of claim 4, wherein the mitochondrially targeted antioxidants are physiological nonenzymatic antioxidants such as vitamin E, ascorbic acid, glutathione and NADPH, nonenzymatic naturally occurring antioxidants of herbal origin such as resveratrol, enzymatic antioxidants such as superoxide dismutases and catalase as well as synthetic antioxidants such as modified tocopherols, derivatives of stobadine and dihydropyridines and modified enzymatic antioxidants.

8. A method for treating an amyloid protein disease by administering a nanoliposome composition comprising lipids or phospholipids that attach to amyloid proteins.

9. The method of claim 8, wherein the amyloid protein disease is light chain amyloidosis, alzheimer's disease, diabetes mellitus, parkinson's disease, amyotrophic lateral sclerosis, huntington's disease, AA amyloidosis, ATTR amyloidosis, hemodialysis associated amyloidosis (beta 2 microglobulin), a prion disease, Creutzfeld Jakob disease, bovine spongiform encephalopathy, scrapie, finish type amyloidosis, cerebral amyloid angiopathy, prolactinoma, familial corneal amyloidosis, senile amyloid of atria, medullary carcinoma of thyroid, or an amyloid form of atherosclerosis.

10. The method of claim 8, wherein the nanoliposomes are attached to amyloid proteins.

11. The method of claim 8, wherein the nanoliposomes reduce the amount of soluble amyloid proteins.

12. The method of claim 8, wherein endothelial function of human arterioles exposed to amyloid proteins is preserved by co-treating with nanoliposomes.

13. The method of claim 8, wherein endothelial cells exposed to amyloid proteins are protected against cell damage by co-treating with amyloid proteins.

14.-15. (canceled)

16. The method of claim 8, wherein the lipids or phospholipids that attach to amyloid proteins are composed of cholesterol, phosphatidylcholine, phosphatidic acid, sphingomyelin, GM1 ganglioside, STTP, other phospholipids or a mixture thereof.

17. The method of claim 8, further comprising one or more cargoes comprising apolipoproteins, peptides, antibodies, nucleic acids, aptamers, mitochondrially targeted antioxidants or mixtures thereof.

18. The method of claim 17, wherein the apolipoprotein is clusterin, apolipoprotein A1, or E.

19. The method of claim 17, wherein the nucleic acid is siRNA.

20. The method of claim 17, wherein the mitochondrially targeted antioxidants are physiological nonenzymatic antioxidants such as vitamin E, ascorbic acid, glutathione and NADPH, nonenzymatic naturally occurring antioxidants of herbal origin such as resveratrol, enzymatic antioxidants such as superoxide dismutases and catalase as well as synthetic antioxidants such as modified tocopherols, derivatives of stobadine and dihydropyridines and modified enzymatic antioxidants.

21. A method for treating amyloid protein disease by administering a nanoliposome composition comprising lipids or phospholipids that attach to amyloid proteins to reduce tissue injury caused by amyloid proteins to vascular tissue and other organs.

22. The method of claim 21, wherein the amyloid protein disease is light chain amyloidosis.
